(19) United States
(12) Patent Application Publication
(10) Pub. No.: US 2024/0335791 A1
(43) Pub. Date: Oct. 10, 2024

(54) MULTI-STAGE MEMBRANE FILTRATION SYSTEM AND METHOD FOR OPERATING A MULTI-STAGE MEMBRANE FILTRATION SYSTEM

(71) Applicant: B. Braun Avitum AG, Melsungen (DE)

(72) Inventors: Nino Lieb, Melsungen (DE); Philipp Odernheimer, Melsungen (DE); Jonas Giesa, Kassel (DE); Stephan Krietemeyer, Kandern (DE)

(21) Appl. No.: 18/625,360

(22) Filed: Apr. 3, 2024

(30) Foreign Application Priority Data
Apr. 5, 2023 (DE) 10 2023 108 726.6

(51) Int. Cl. B01D 61/02; B01D 61/08

(52) U.S. Cl. CPC B01D 61/026; B01D 61/08; B01D 2311/16; B01D 2311/25; B01D 2313/083; B01D 2313/18; B01D 2313/243; B01D 2313/54; B01D 2313/60; B01D 2313/70; B01D 2313/903; B01D 2317/02

(57) ABSTRACT

A membrane filtration system, in particular for reverse osmosis, includes a plurality of stages. Each stage has at least one pump and one filter membrane. The stages are hydraulically interconnected in sequence by connecting lines. The system also includes a bypass line for each stage for diverting liquid flow around the stage. At least one bypass valve blocks the respective bypass line in the closed state. The system further includes a control and regulating unit that can actuate the respective bypass valve.

### CROSS-REFERENCE TO RELATED APPLICATION(S)

This application claims priority under 35 U.S.C. § 119 to German Patent Application No. 10 2023 108 726.6, filed on Apr. 5, 2023, the content of which is incorporated by reference herein in its entirety.

### FIELD

The disclosure relates to a membrane filtration system, in particular for reverse osmosis, comprising a plurality of stages, wherein each stage comprises at least one pump and one filter membrane, and wherein the stages are hydraulically connected to one another in sequence by connecting lines, and wherein a bypass line is provided for each stage for bypassing the liquid flow around this stage, and wherein at least one bypass valve is provided which blocks the respective bypass line in the closed state, It further relates to a method for operating the membrane filtration system.

### BACKGROUND

In known multi-stage membrane filtration systems with several stages, each comprising at least one filter membrane and at least one pump for pumping liquid through this filter membrane, the permeate from the previous stage is fed into the subsequent stage. If a stage fails completely or is not functional, the liquid flow can be fed directly into the next stage by manually actuating a corresponding valve, bypassing the defective stage.

A disadvantage of the known membrane filtration systems or membrane filter systems described is that the corresponding valve must be operated manually. Until the valve or valves are switched, the supply to the connected consumers cannot be guaranteed. Incorrect valve positions can cause unfiltered water to flow into the ring main, which can endanger patients during dialysis therapy, for example.

### SUMMARY

The disclosure is therefore based on the object of improving a membrane filtration system in such a way that, in the event of a stage failure, the supply to the connected consumers is ensured and the deactivation of the failed stage takes place reliably. Furthermore, an improved process for membrane filtration is to be specified.

With regard to the membrane filtration system, this object is solved in accordance with the disclosure in that a control and regulation unit is provided which is configured to control the respective bypass valve.

The disclosure is based on the consideration that in multi-stage systems, if one stage is damaged or fails, the supply to the consumers should be ensured with as little interruption as possible. This is particularly important when the system is used for dialysis therapy.

As has now been recognized, these requirements can be met by compensating for the failure of a filter stage by initiating emergency operation in multi-stage membrane filter systems that filter the permeate several times. For this purpose, the defective or non-functional part of the system (hydraulic components and/or sensors) is bypassed by a flow detour within the membrane filter system or the membrane filtration system.

The main advantages of using a double-stage system are reliability and regulatory requirements (some countries require double-filtered water for use in hemodialysis).

The expression that the control and regulation unit is configured to actuate the bypass valves means in particular that the control and regulation unit is adapted or designed or programmed or set up to perform the actuation. The corresponding control is preferably implemented in hardware and/or software in the control and regulation unit.

Advantageously, the control and regulation unit is configured to open the bypass valve assigned to the respective stage in an emergency operation with at least one defective stage, so that the fluid is diverted around the defective stage. The bypass line guides the fluid past the defective stage to the hydraulically connected or next stage.

In a preferred embodiment, a bypass valve is arranged in each bypass line. In a two-stage system, two bypass lines are therefore provided, in each of which a bypass valve is arranged.

Advantageously, a check valve is arranged in the bypass line, in particular upstream of the respective bypass valve in the bypass line, which blocks the backflow through the bypass valve. Check valves are particularly suitable for preventing backflow, as their simple mechanical design means they are durable and not prone to faults. In addition, these valves do not require any control logic, as the onset of emergency operation reverses the pressure conditions within the system in such a way that the check valves close automatically. As an alternative to check valves, electrically actuated valves can also be used, which are connected to the aforementioned control and regulation unit.

In a preferred embodiment, at least one bypass valve is designed as a multi-port valve. In a two-stage membrane filtration system, only one valve is required in this way to hydraulically bypass the first or second stage as required. Only one signal connection from the control and regulation unit to the multi-port valve is also required, which saves material and simplifies the configuration of the system.

Advantageously, the control and regulation unit is configured to receive sensor data and/or process data from the membrane filtration system on the signal input side. In this way, the control and regulation unit is enabled to monitor the system and to recognize and react to malfunctions, in particular the failure/defect or malfunction of a stage. The control unit of the membrane filter system recognizes the malfunction and automatically initiates emergency operation, thus avoiding system downtimes and preventing incorrect user interactions (e.g. valve malpositions).

In emergency mode, the control and regulation unit opens the bypass valve assigned to the respective stage so that the liquid is diverted around the defective stage if the sensor data and/or process data received is outside a predefined threshold range, in particular if a zero signal from a sensor is present.

The sensors can be continuously monitored if they emit a “minimum signal” (e.g. 4 mA) to enable the difference between the measured 0 value and a defect to be detected. The sensors and/or process values can be checked for functionality using logical statements. For example: IF operating mode “On” THEN volume flow rate stage 1 GREATER than 3001/h. If this condition is no longer true, a defect in the first stage can be triggered. The logical statement is exemplary and can be extended as required with logically linked sensor combinations and process values. This extension can be carried out using logic operators (AND/OR/NOT etc.).

The membrane filtration system preferably comprises at least one pressure sensor and/or at least one volume flow sensor or flow sensor, whereby the respective sensor is connected to the control and regulation unit on the signal input side for the transmission of sensor data. In this way, the states of the components of the system can be detected. Preferably, each of the stages comprises a pressure sensor and/or at least one volume flow sensor or flow sensor, so that the pressure and volume flow can be recorded separately for each of the stages, which improves the targeted detection of a failure or malfunction of a stage. In addition to recording and evaluating sensor data, binary signals can also be evaluated in order to draw conclusions about major failures. For example, fuses can be evaluated and the triggering of fuses can be correlated to the failure of stages or individual pumps.

The at least one pressure sensor is preferably a pressure sensor for measuring the fill level of a tank, in particular the holding tank of the membrane filtration system, and/or the at least one volumetric flow sensor is preferably a volumetric flow sensor for measuring the concentrate flow and/or the at least one volumetric flow sensor is a volumetric flow sensor for measuring the permeate flow.

The membrane filtration system preferably comprises at least one pump, at least one filter membrane and at least one automatic circuit breaker and/or at least one circuit breaker, which is connected to the control and regulation unit on the signal input side for the transmission of process data. The membrane filtration system comprises two or more stages. The respective stage preferably comprises a booster pump, which presses the liquid fed into the stage in an inlet flow into a membrane module. The membrane module comprises one or more membranes, each of which is connected in groups in series or in parallel. Several membrane modules can be connected in series to each other in a stage.

The process produces a filtered permeate flow in a permeate line starting from the membrane module and a concentrate flow in a concentrate line, which transports the retained substances away. The respective stage therefore preferably comprises a circulation pump, which conveys part of the concentrate, i.e. the recirculated concentrate, in a concentrate flow back to the booster pump, where it mixes with the feed flow and repeats the process as feed water. The non-recirculated portion of the concentrate is discharged from the membrane filtration system via a valve (e.g. a solenoid valve, needle valve, electric control valve, etc.).

The control and regulation unit can receive and evaluate information about the operating status of important components, in particular in addition to the pressure and volume ratios, thereby improving the accuracy and reliability of recognizing or detecting a failed or improperly functioning stage.

The connection on the signal input side, i.e. the connection between sensors and/or frequency converters and/or automatic circuit breakers and/or circuit breakers to the control and regulation unit, can be analog, digital via electrical transmission lines and/or via a bus.

In a preferred embodiment, the control and regulation unit opens at least one bypass valve in emergency mode if the sensor and/or process data exceeds or falls below at least one threshold value. In particular, the control and regulation unit assigns this data to one or more stages, which are thus recognized as defective or non-functional and switched off. The corresponding stage is hydraulically bypassed by the bypass valve. In multi-stage systems with three or more stages, several bypass valves can be opened so that the corresponding stages are hydraulically bypassed.

Advantageously, the control and regulation unit controls at least one pump in emergency mode in such a way that process inlet water continues to be pressed through at least one intact membrane module. This ensures that the consumers can continue to be supplied with permeate if a stage fails.

The control and regulation unit is advantageously configured to permanently monitor the components of the membrane filtration system that output continuous signals, in particular pressure sensors and/or volume flow sensors, and to test the components of the membrane filtration system that do not output continuous signals, in particular solenoid valves, in a component test. The component test generates stimuli in the system that must fulfill an expected result. If this result does not meet the defined expectations, the tested component is considered defective.

The control and regulation unit is preferably configured to apply parameters of the membrane filtration system and/or the water yield in an emergency mode.

The parameters are preferably control parameters and/or setpoints and/or process variables and/or water yield. The control and regulation unit preferably uses these parameters to carry out error/alarm/information handling (i.e. actions caused by events), operating characteristics (pump start/stop sequence) and/or interactions with connected peripheral devices.

In multi-stage systems, different parameters can be used for each stage. In emergency operation, depending on the emergency operation, the parameters of the defective stage must be partially or fully applied to the compensation stage in order to maintain the same quality of the process. It is also possible that the control and regulation unit controls other sensors and actuators, as the hydraulic process has changed during emergency operation.

The purpose of adjusting or applying the parameters is to be able to carry out the override mode reliably. For example, calculations (controller/control ratio calculation) have to be adjusted to ensure that the correct/adjusted process variable is used and the correct system behavior is achieved and also that the new system configuration (e.g. quality of the process inlet water) is taken into account.

In a preferred embodiment, at least one bypass valve is designed as a solenoid valve. This corresponds to a cost-effective valve design. Due to its mode of operation, this valve also does not offer an undefined state, such as a partially open valve, as would be the case with disk valves or electric control valves, for example, where an intermediate position is possible. As an alternative, control valves or motor control valves can be used, which open partially or fully when actuated.

The respective connecting line connects a hydraulic outlet of a stage to an inlet of the hydraulically downstream stage, whereby a non-return valve is advantageously arranged in at least one outlet of a stage, which prevents backflow into this stage. In a preferred embodiment, each of these stage outlets has a non-return valve.

The control and regulation unit is preferably configured to check the functionality of the bypass valves and/or sensors in a test mode before regular operation of the membrane filtration system. In this way, possible defects or malfunctions can be detected and rectified before regular operation of the system. This reduces the likelihood of malfunctions during operation of the system, thus ensuring uninterrupted patient care during dialysis.

A preferred embodiment of the membrane filtration system comprises exactly two stages.

With regard to the method, the above-mentioned task is solved in accordance with the disclosure in that the operation of the membrane filtration system is monitored essentially continuously by a control and regulation unit, and wherein, if a malfunction of a stage is detected, the control and regulation unit at least one bypass valve connected in a bypass line opens, providing a hydraulic bypass of this stage

Bridging is preferably achieved by opening bypass lines so that the liquid is diverted around the respective stage with a malfunction.

A malfunction is preferably detected by monitoring sensors, as discussed above in connection with the membrane filtration system. The switchover to emergency operation takes place automatically via the integrated control or control and regulation unit; no manual user interaction is required. Advantageously, continuous and/or discontinuous monitoring of system components of the membrane filtration system is carried out to detect this state of malfunction.

The bypass lines or bypass lines are used exclusively for the bypassing of failed components. Simultaneous switching of both bypass lines is preferably prevented so that no process inlet water enters the ring main, as this can lead to patient injury.

The advantages of the disclosure lie in particular in the fact that an automatic bypass of a stage recognized as defective enables a rapid response to the new situation, so that a continuous supply to the consumer can be achieved. This is particularly relevant when using the membrane filtration system for reverse osmosis in dialysis, as an inadequate supply can endanger the health of patients.

### BRIEF DESCRIPTION OF THE DRAWINGS

An embodiment of the disclosure is explained in more detail with the aid of the following drawing figures, which provide a highly schematized representation.

FIG. 1 shows a multi-stage membrane filtration system according to the state of the art.

FIG. 2 shows a two-stage membrane filtration system in a first preferred embodiment with solenoid valves.

FIG. 3 shows a two-stage membrane filtration system in a second preferred embodiment with a multi-port valve.

FIG. 4 shows configurations of the multi-port valve of the membrane filtration system according to FIG. 3.

FIG. 5 shows a two-stage membrane filtration system with more than two stages.

FIG. 6 shows a stage of a membrane filtration system.

FIG. 7 shows a first stage of a membrane filtration system with sensors.

Identical parts are marked with the same reference signs in all figures.

### DETAILED DESCRIPTION

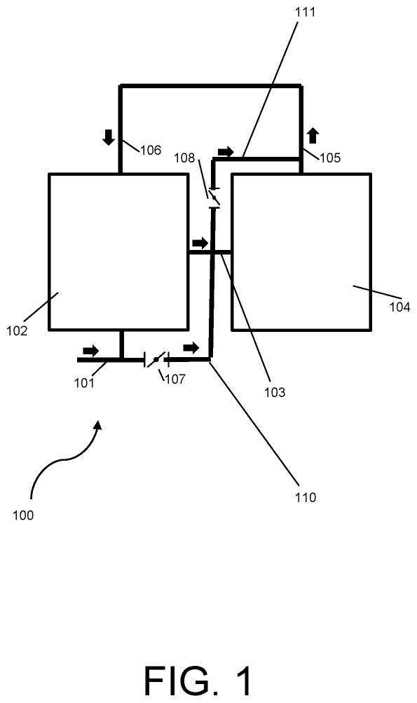

A membrane filtration system 100 of the prior art is shown in FIG. 1. Regularly, the process input water 101 flows into the first stage 102 of the membrane filtration system 100. This stage 102 comprises at least a pump and a filter membrane. The permeate generated in this stage 102 is fed into the second stage 104 via a connecting section or connecting line 103. This also consists of at least one pump and one filter membrane. After the second filtration, the permeate is fed into the ring main flow 105. Water that is not removed from the circuit is fed back into the system via the ring line return 106. In the event of a malfunction of the first stage 102, a valve 107 can be opened manually, provided the second stage 104 is intact, which directs the input process water 101 through a bypass line 110 into the second stage 104.

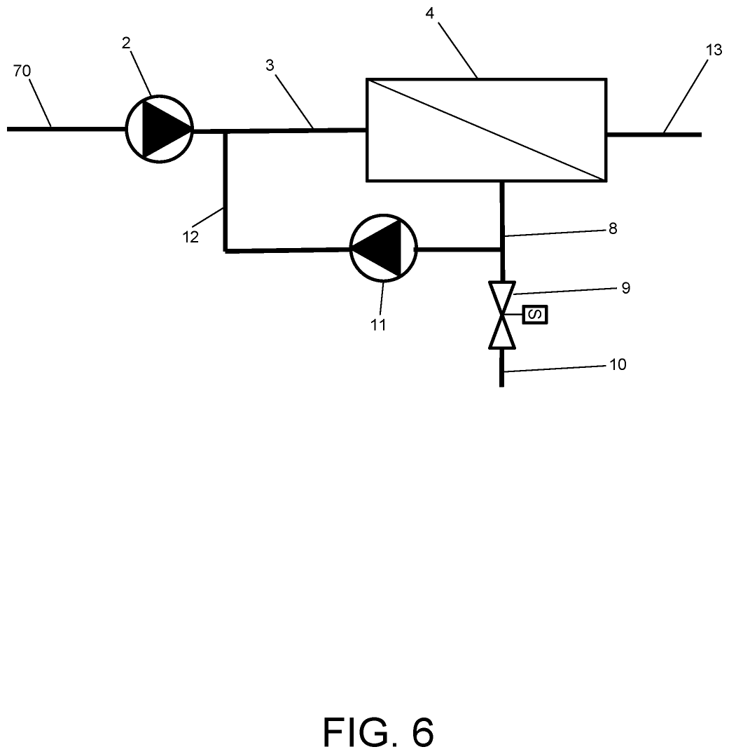

The preferred hydraulic structure of a stage 102, 104 of the membrane filter system 100 shown in FIG. 1 is illustrated in FIG. 6. A feed flow 70 is forced into a membrane module 4 via a booster pump 2. The membrane module 4 may contain one or more membranes, each of which is connected in groups in series or in parallel. Furthermore, several membrane modules 4 can be connected in series with one another.

The process produces a filtered permeate flow in a permeate line 13 starting from the membrane module 4 and a concentrate flow in a concentrate line 8, which removes the retained substances. A circulation pump 11 conveys part of the concentrate, i.e. the recirculated concentrate 12, in the concentrate flow back to the booster pump 2, where it mixes with the feed flow 70 and repeats the process as feed water. The non-recirculated portion of the concentrate is fed via a valve 9 (e.g. a solenoid valve, needle valve, electric control valve, etc.) into the hydraulically downstream stage or in the last stage as wastewater 10 from the membrane filtration system 100.

If the second stage 104 malfunctions and the first stage 102 is intact, a valve 108 can be opened manually so that the permeate can flow through a bypass line 111 into the ring line feed 105. During this period, the membrane filtration system 100 produces single-filtered permeate instead of multi-filtered permeate. The product is fed into the ring line via the feed line 105. The disadvantage of this solution is that the valves 107, 108 must be operated manually. Until the valves 107, 108 are switched, a supply to the connected consumers cannot be guaranteed. A certain level of system knowledge is also required from the operator. Incorrect valve positions can cause unfiltered water to flow into the ring main, which, for example, can endanger patients during dialysis therapy.

In further developments of the membrane filtration system 100 of the stand, a solenoid valve can be used to bypass the second stage of the system. The bypass requires manual interaction with the user, and only emergency operation of the first stage 102 can be realized using this method.

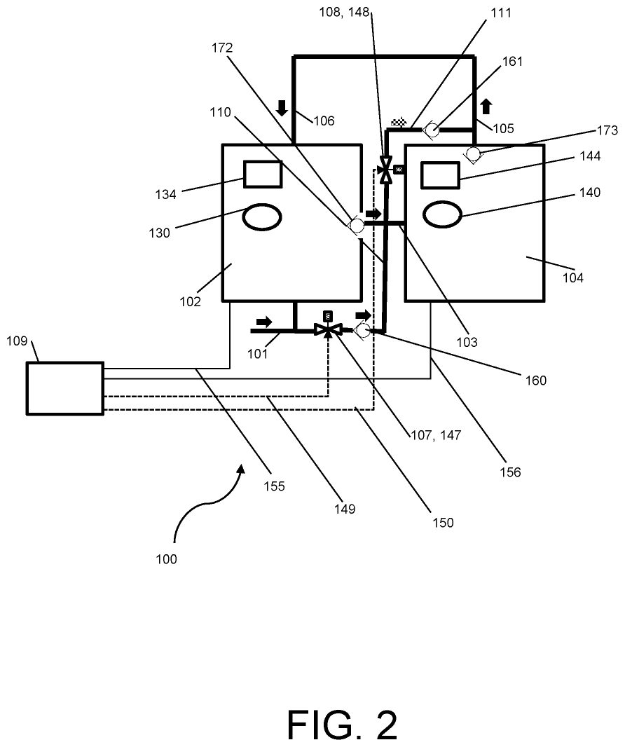

A preferred embodiment of a membrane filtration system 100 according to the disclosure is shown in FIG. 2. Also in the embodiments of the membrane filtration system 100 according to FIGS. 2, 3 and 5, the preferred hydraulic structure of a stage 102, 104 is shown in FIG. 6.

Regularly, the process input water 101 flows into the first stage 102 of the membrane filtration system 100. This stage 102 comprises at least one pump 130 and at least one filter membrane 134. The filter membrane 134 can be arranged in a membrane module. Several filter membranes can be arranged in series and/or in parallel in the membrane module.

The permeate generated in this stage 102 is fed into the second stage 104 via a connecting section or connecting line 103. This second stage 104 also comprises at least one pump 140 and a filter membrane 144. The filter membrane 144 can be arranged in a membrane module. Several filter membranes can be arranged in series and/or in parallel in the membrane module.

After the second filtration, the permeate is fed into the ring line feed 105. Water that is not removed from the circuit is returned to the system via the ring line return 106. As already described in connection with FIG. 1 regarding the prior art, the first stage 102 can be bypassed by directing the process input water 101 through the bypass line 110 via a valve 107 to the second stage 104. The second stage 104 can be bypassed by directing the permeate of the first stage 102 through the bypass line 111 via a valve 108 into the feed 105. The two valves 107, 108 are designed in the present case as solenoid valves 147, 148.

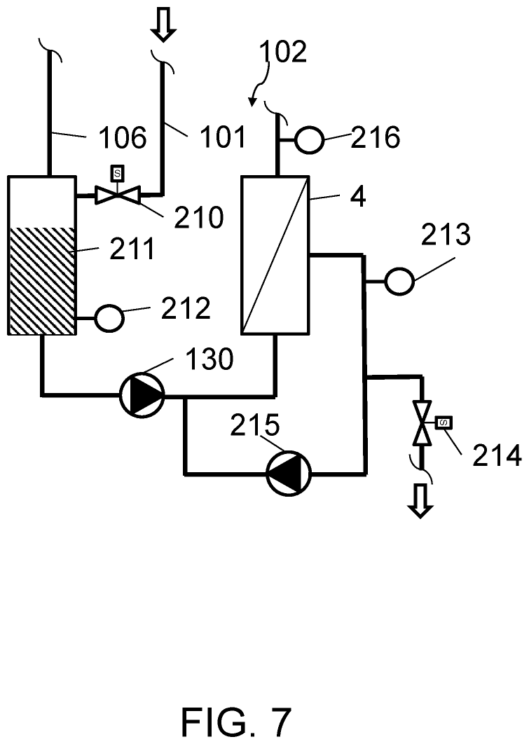

An illustration of the hydraulic first stage 102 of a membrane filtration system 100 with sensors is shown in FIG. 7. The stage 102 has a pressure sensor 212 that measures the pressure in the tank 211 of the membrane filtration system 100. A volumetric flow sensor 213 measures the concentrate flow that flows out of the membrane module 4. A further volumetric flow sensor 216 measures the permeate flow flowing out of the membrane module 4.

The emergency operating solenoid valves or solenoid valves 147, 148 are switched by a control and regulation unit 109, which is connected to the solenoid valve 147 via a first control line 149 and to the second solenoid valve 148 via a second control line 150. The control and regulation unit 109 is designed in terms of hardware and/or software, in particular it is a combination of software and hardware and is a component of the membrane filtration system 100.

The control and regulation unit 109 uses data from the respective stages 102, 104 to assess whether emergency operation is required. For this purpose, the control and regulation unit 109 is connected on the signal input side via a first signal line 155 to components of the first stage 102 and via a second signal line 156 to components of the second stage 104. The two signal lines 155, 156 are shown schematically and can represent several signal lines or a bus. The data transmitted to the control and regulation unit 109 via the control lines 155, 156 comprise sensor values and process data. The sensor values are transmitted by pressure or volumetric flow sensors or flow sensors (not shown). The process data is transmitted by frequency converters (if present) of the at least two pumps 130, 140 and automatic circuit breakers (not shown).

The control and regulation unit 109 compares the transmitted sensor values or sensor data and process data with predefined threshold values or threshold ranges. If one or more sensor or process data lie outside the threshold ranges or exceed or fall below the threshold values, the control and regulation unit 109 deactivates the stage 102, 104 recognized as defective or non-functional in an emergency mode. A component is recognized as defective if the exceeding or falling below of the threshold values occurs for a predetermined period of time. A stage 102, 104 is considered defective if one or more components declared as functionally critical are defective.

The activation of emergency operation by the control and regulation unit 109 is described below as an example. The following faults can occur in the membrane filtration system 100. In the event of a sensor error, a monitored sensor is defective and causes emergency operation to be activated. In the event of an actuator fault, a monitored actuator, in particular a pump 130, 140, 215, is defective and activates emergency operation.

Chaining errors can occur. For example, emergency operation can be activated by a monitored sensor and a monitored actuator by the control and regulation unit 109.

Emergency operation can also be activated by a monitored sensor if it is below a defined level for a time interval during an operating mode (monitored by the control and regulation unit 109).

A sensor error is present, for example, if the signal of the tank level sensor, i.e. the signal of the pressure sensor 212 (see FIG. 7) is unreliable, in particular if it is outside a predefined threshold value range or if the pressure sensor 212 fails completely and no longer supplies a signal. In this case, monitoring of the tank filling level of the tank 211 is no longer possible and the stage 102, 104 can no longer be operated reliably. In this case, the control and regulation unit 109 switches the valve 107 in such a way that the defective stage 102 is bypassed.

An actuator fault is present, for example, if the pump 130 is defective or the pump 130 or the frequency converter optionally connected to it has failed. In this case, the reverse osmotic pressure can no longer be generated and the corresponding stage 102 cannot generate any permeate. In this case too, the control and regulation unit 109 switches the valve 107 in such a way that the defective stage 102 is bypassed.

As an example of a chaining error, this may be present in the circulation path (membrane model 4 via pump 215) of the first stage 102. The circulation pump 215 is active and the volume flow sensor 213 indicates a value that is too low (e.g.: 0 l/h). The effect of this is that the corresponding membrane module 4 is no longer overflowed and accelerated wear of the module occurs. This can be caused by blocked pipe sections or problems with the circulation pump 215.

In another example of a chaining error, the permeate production of the first stage 102 may be faulty. The membrane filtration system 100 is in “production mode” (monitored by control and regulation unit 109) and the permeate volume flow of the first stage 102 (detected via volume flow sensor 216) is too low (e.g.: 0 l/h) for a period of time that is longer than a predetermined period of time, for example 5 seconds. The effect of this error is that the first stage 102 no longer delivers any permeate because there is a pump defect and/or there is a line defect and/or there is no more process inlet water.

In both cases, the control and regulation unit 109 switches the valve 107 in such a way that the defective stage 102 is bypassed.

The central control and regulation unit 109 or control and evaluation unit can thus ensure that the bypassed stage 102, 104 is switched off and the correct valves 107, 108 are switched. Accidental switching of the wrong valve 107, 108 or simultaneous switching of both valves 107, 108 can also be prevented. The control and evaluation unit 109 is a combination of software and hardware.

If technically necessary or advantageous, check valves 160, 161 or functionally equivalent components (non-return valves, overflow valves) are installed upstream or downstream of the solenoid valves 147, 148 to prevent an inverse flow (due to the process pressure). A combination of solenoid valve 147, 148 and check valve is then required. Accordingly, a check valve 160 is arranged upstream of the valve 107, which blocks the backflow of liquid through the valve 107. A further check valve 161 is arranged upstream of the valve 108, which blocks the return flow of liquid through the valve 108. In addition to triggering emergency operation during ongoing operation, emergency operation can also be started before the actual operation is started by testing whether sensors or actuators are defective.

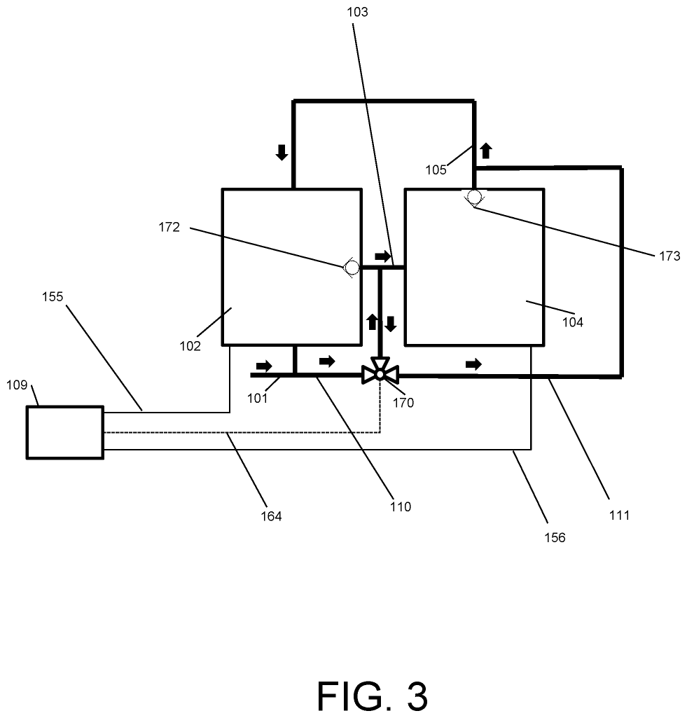

In the embodiment variant shown in FIG. 3, check valves upstream of the multi-port valve 170 are not necessary. Generally and preferably, however, the individual stages 102, 104 each have a check valve 172, 173 at their outlets in order to prevent an inverse volume flow through the stage.

A further preferred embodiment of a membrane filtration system 100 according to the disclosure is shown in FIG. 3. Also in this embodiment, the first stage 102 comprises at least one pump (not shown) and at least one filter membrane (not shown) and the second stage 104 comprises at least one pump (not shown) and one filter membrane (not shown), wherein membrane modules with filter membranes connected in series or in parallel may be provided in each case. In particular, the two stages 102, 104 are designed as described in connection with FIG. 6 above, i.e. they each comprise a booster pump 2, a filter module 4 and a circulation pump 11.

In contrast to the embodiment shown in FIG. 2, the solenoid valves 147, 148 are replaced by a multi-port valve 170, which is actuated by the control and regulation unit 109 via a control line 164. An unauthorized configuration of this valve can be implemented, for example, via an L-bore in the valve. The multi-port valve 170 can bypass the first stage 102 by directing the process input water 101 through the bypass line 110 into the connecting line 103 or into the second stage 104. The second stage 104 can be bypassed by directing the permeate of the first stage at the connecting line 103 via the multi-port valve 170 through the bypass line 111 into the flow 105. The invalid path that the process inlet water 101 is routed into the flow 105 via the multi-port valve 170 is excluded by the geometry of the valve.

Also in the embodiment variant shown in FIG. 3, check valves upstream of the multi-port valve 170 are not necessary. Generally and preferably, however, the individual stages 102, 104 each have a check valve 172, 173 at their outlets in order to prevent an inverse volume flow through the stage.

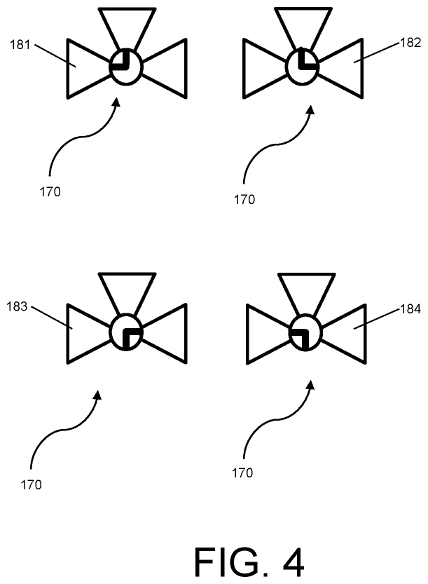

In FIG. 4, the exemplary positions of the multi-port valve 170 are shown. Here, the configuration 181 (top left in FIG. 4) describes the bypassing of the first stage 102, the configuration 182 describes the bypassing of the second stage 104, and the configurations 183, 184 each represent the normal state, i.e. without bypassing.

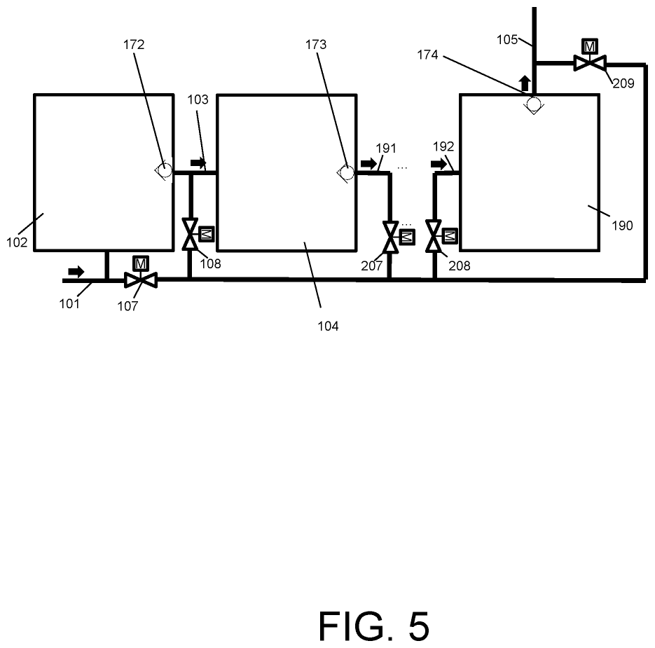

In FIGS. 2 and 3, two-stage membrane filtration systems 100 are described. The inventive idea is scalable to membrane filtration systems 100 with more than two stages 102, 104. An N-stage (N>2) system is shown generically and schematically in FIG. 5, whereby the control and regulation unit 109 is not shown here. In this N-stage system, the process input water 101 enters the first stage 102 during normal operation. The product of the first stage 102 is fed into the second stage 104 via a connecting line 103. The general procedure is continued until the Nth stage 190, after which the water is fed into the line to the feed 105. Should the first stage 102 fail, the process input water 101 can be transported via the valve 107 to the second stage 104 or alternatively to any stage by opening the corresponding valve 108, 207 or 208.

If a stage within the system fails, e.g. the second stage 104, this stage 104 can be bypassed by opening the upstream valve (here valve 108) and the downstream valve (here valve 207). The defective stage 104 is thus hydraulically excluded from the system. If the last stage 190 fails, this stage can be hydraulically excluded from the system via the upstream valve 208 and the valve 209 connected to the line to the consumers 220.

In the embodiment variant shown in FIG. 3, check valves upstream of the multi-port valve 170 are not necessary. Generally and preferably, however, the individual stages 102, 104, 190 each have a check valve 172, 173 at their outlets in order to prevent an inverse volume flow through the stage.

The valves 107, 108, 209, . . . can be controlled via a central control unit. Multi-port valves (see FIG. 3) can also be used instead of motor control valves, solenoid valves or similar.

### LIST OF REFERENCE SIGNS

2 booster pump
4 membrane module
8 concentrate line
9 valve
11 circulation pump
12 recirculated concentrate
13 permeate line
70 inlet flow
100 membrane filtration system
101 process input water
102 first stage
103 connecting line
104 second stage
105 ring line feed
106 ring line return
107 valve
108 valve
110 bypass line
111 bypass line
130 pump
134 filter membrane
140 pump
144 filter membrane
147 solenoid valve
148 solenoid valve
149 first control line
150 second control line
155 first signal line
156 second signal line
160 check valve
161 check valve
164 control line
170 multi-port valve
172 check valve
173 check valve
174 check valve
181 configuration
182 configuration
183 configuration
184 configuration
190 n-th stage
191 connecting line
192 connecting line
198 stage
207 valve
208 valve
209 valve
210 valve
211 tank
212 pressure sensor
213 volume flow sensor
214 valve
215 pump
216 volume flow sensor

1. A membrane filtration system comprising:
a plurality of stages;
at least one bypass valve; and
a control and regulating unit,
a bypass line for each stage for bypassing liquid flow,
each stage comprising at least one pump, at least one filter membrane, and a bypass line for bypassing liquid flow around said stage,
the stages being hydraulically connected to one another in sequence by connecting lines,
the at least one bypass valve blocking the respective bypass line in a closed state, and
the control and regulating unit being configured to control the respective bypass valve.

2. The membrane filtration system according to claim 1, wherein the control and regulation unit is configured to open the bypass valve associated with the respective stage in an emergency operation with at least one defective stage, so that the liquid is diverted around the defective stage.

3. The membrane filtration system according to claim 1, wherein the at least one bypass valve comprises a bypass valve arranged in each bypass line.

4. The membrane filtration system according to claim 1, wherein the control and regulation unit is configured to receive sensor data and/or process data of the membrane filtration system on a signal input side.

5. The membrane filtration system according to claim 4, wherein the control and regulation unit opens the bypass valve associated with the respective stage in an emergency mode, so that the liquid is diverted around said respective stage when one of the received sensor data and/or process data is outside a predetermined threshold range.

6. The membrane filtration system according to claim 4, wherein the membrane filtration system comprises at least one pressure sensor and/or at least one volume flow sensor, wherein the respective sensor is connected to the control and regulation unit on the signal input side for transmission of sensor data.

7. The membrane filtration system according to claim 6, wherein the at least one pressure sensor is a pressure sensor for measuring a fill level of a tank, and/or wherein the at least one volume flow sensor is a volume flow sensor for measuring a concentrate flow and/or wherein the at least one volume flow sensor is a volume flow sensor for measuring a permeate flow.

8. The membrane filtration system according to claim 6, wherein the membrane filtration system comprises at least one frequency converter of a pump and/or at least one automatic circuit breaker and/or at least one circuit breaker, which is connected to the control and regulation unit on the signal input side for the transmission of process data.

9. The membrane filtration system according to claim 8, wherein the signal input side comprises a signal input-side connection that is analog, digital via electrical transmission lines and/or via a bus.

10. The membrane filtration system according to claim 8, wherein the control and regulation unit opens at least one bypass valve in an emergency mode when at least one threshold value of the sensor data and/or the process data is exceeded or not reached.

11. The membrane filtration system according to claim 10, wherein the control and regulation unit controls at least one pump in emergency operation in such a way that process input water continues to be pressed through at least one intact membrane module.

12. The membrane filtration system according to claim 1, wherein the control and regulation unit is configured to permanently monitor components of the membrane filtration system which output continuous signals and to test the components of the membrane filtration system which do not output continuous signals.

13. The membrane filtration system according to claim 1, wherein the control and regulation unit is configured to apply parameters of the membrane filtration system in an emergency mode.

14. The membrane filtration system according to claim 13, wherein the parameters are control parameters and/or setpoints and/or process variables and/or a water yield.

15. The membrane filtration system according to claim 1, wherein at least one bypass valve is designed as a solenoid valve.

16. The membrane filtration system according to claim 1, wherein the control and regulation unit is configured to check the bypass valves and/or sensors for their functionality in a test mode prior to a regular operation of the membrane filtration system.

17. The membrane filtration system according to claim 1, wherein the plurality of stages consists of two stages.

18. A method for operating a membrane filtration system having a plurality of stages that are hydraulically adjoined, wherein liquid is passed successively through the stages in each of which the liquid is filtered, wherein a permeate of a respective previous stage is conveyed into a respective hydraulically following stage, the method comprising the steps of:
continuously monitoring operation of the membrane filtration system by a control and regulating unit;
opening at least one bypass valve connected in a bypass line by the control and regulating unit when a malfunction of a stage is detected to hydraulically bypass the stage.
